(19) United States
(12) Patent Application Publication
(10) Pub. No.: US 2015/0159179 A1
(43) Pub. Date: Jun. 11, 2015

(54) METHOD FOR PRODUCING BIOFUELS AND FOOD CO-PRODUCTS USING EXTRACTS OF MICROALGAE CULTURES

(71) Applicants: Francisco Manuel Reyes Sosa, Sevilla (ES); Maria Piera Alberola, Sevilla (ES); Maite Zazpe Cenoz, Sevilla (ES); Javier Iruretagoyena Aldaz, Sevilla (ES)

(72) Inventors: Francisco Manuel Reyes Sosa, Sevilla (ES); Maria Piera Alberola, Sevilla (ES); Maite Zazpe Cenoz, Sevilla (ES); Javier Iruretagoyena Aldaz, Sevilla (ES)

(73) Assignee: ABENGOA BIOENERGÍA NUEVAS TECNOLOGIAS, S.A., Sevilla (ES)

(21) Appl. No.: 14/402,295

(22) PCT Filed: Jul. 9, 2012
(86) PCT No.: PCT/ES2012/070513

§ 371 Date: Feb. 27, 2015

(30) Foreign Application Priority Data
Jun. 6, 2012 (ES) P201230880

(51) Int. Cl. C12P 7/14; A23L 1/105

(52) U.S. Cl. CPC C12P 7/14; A23L 1/105; A23V 2002/00

(57) ABSTRACT

The present invention relates to a method for the production of biofuels and food co-products which, thanks to the addition of microalgae culture extracts, optimizes the production of biofuel, in terms of time and yield, and improves the protein composition of its co-products useful in the food industry, as compared to similar methods for the production of biofuel and co-products which do not use microalgae culture extracts.

The present invention falls within the field of biofuels; it specifically relates to a method for the production of biofuels, preferably ethanol or bioethanol, and food co-products from fermentable plant biomass, wherein a microalgae culture extract is used before or after the fermentation process.

### STATE OF THE ART

Conventionally, ethanol is produced by a chemical synthesis process in which ethanol is synthesized from fossil sources, such as coal or oil, via ethylene, or by means of a fermentation process arising from biomass such as sugarcane, corn or starch, thanks to the action of microorganisms such as yeasts. In general, the latter way of ethanol production is a biological process for the production of CO₂, ethanol and a co-product rich in protein and recoverable as animal feed (DDGs “Dried Distillers Grains with Solubles”).

Currently, the technology regarding microalgae production for biofuels, and to a lesser extent, bioethanol (EP0645456, WO2008105618), has undergone development. Thus, some methods using starch from microalgae as a starting material have been proposed for the production of ethanol. Some of these microalgae contain a large amount (more than 50% by dry weight) of starch and glycogen and therefore are useful as starting material for the production of ethanol, for example, those belonging to the genus Chlorella, Dunaliella, Chlamydomonas, Scenedesmus or Spirulina.

According to the literature, ethanol production using starch from microalgae as a starting material is carried out according to the following procedure: in a first step, the microalgae are cultured photoautotrophically in the presence of light such that they assimilate CO₂ by photosynthesis, or they are cultured heterotrophically in the dark and in the presence of organic materials such as sugars and organic acids. In a second step, since the microalgae store starch or other fermentable polysaccharides inside their cells during growth, this starch is released from them with the aid of mechanical means such as ultrasound or disintegration, or by using enzymes to dissolve the cell walls. Subsequently, the starch is separated by extraction with water or an organic solvent. In a third step, the starch separated by extraction is hydrolyzed to glucose by means of saccharogen amylase enzymes. Finally, the glucose is fermented by adding yeasts capable of carrying out alcoholic fermentation, and thus the glucose is converted into ethanol.

However, there remains a need to develop methods for the production of ethanol useful as biofuel, and its food co-products, which may be more efficient at an industrial level by involving, for instance, an improvement in terms of time and yield, in ethanologenic fermentations from cereal and/or in the enhancement or enrichment of its co-products useful as food.

### DESCRIPTION OF THE INVENTION

The present invention provides a method for the production of a biofuel, preferably ethanol, and food co-products, from now on “method of the invention”, which, thanks to the addition of microalgae culture extracts, optimizes industrial processes of production of biofuels, preferably ethanol, by fermentation, in terms of time and yield, and improves the protein composition of its co-products which are useful in the food industry.

As shown in the examples, the method of the invention improves the times and yields of fermentable plant biomass fermentation for the production of biofuels such as ethanol, as compared to processes that do not use microalgae extracts. In addition, food co-products obtained using this method, preferably DDGs, have higher protein content, giving them great value in the food industry.

Other advantages associated with said method are that the microalgae used in the process are produced in photoautotrophic cultures, in which CO₂ is ultimately assimilated thanks to the energy of light, which implies an advantage since the CO₂ emission in a production plant of biofuels such as ethanol, can be re-used by introducing it back into the process as a nutrient in the microalgae culture. Furthermore, integrating a microalgae culture in a biofuel plant reduces installation and operating costs of the former, as it makes use of streams mainly composed of CO₂ and, therefore, the gas flow volume to be driven is lower. Furthermore, one of the most limiting steps for viable production of microalgae is their harvesting; however, this invention reduces this limitation by using liquid extracts. On the other hand, in the method of the present invention the fermentation stages are accelerated reaching the same or higher yields than in biofuel production processes not using microalgae extracts. This involves the possibility of increasing the production capacity of already active plants, without modifying the process. On the other hand, microalgae production, as occurs with other crops, is variable over the year, but in the present invention, by integrating both processes (biofuel production and microalgae culturing), the plant can keep up the production continuously. Furthermore, microalgae simultaneously represent a nitrogen source in the reaction mixtures, thereby reducing its consumption (urea) during the biofuel production process. Another advantage is that existing biofuel production plants, particularly bioethanol production plants, generate waste or low-cost streams containing nitrogen and phosphorus, which are nutrients for microalgae. Finally, the food co-products, preferably DDGs, obtained in the fermentation, insofar as being enriched with microalgae, have better nutritional qualities.

Based on the foregoing, a first aspect of the invention relates to a method for the production of a biofuel, preferably ethanol, and food co-products from fermentable plant biomass, the “method of the invention”, comprising:
a) preparing an aqueous medium comprising said biomass,
b) simultaneously or sequentially hydrolyzing and fermenting biomass comprised in the aqueous medium of step (a), and
c) distilling the products obtained in step (b) in order to obtain biofuel, preferably ethanol, and food co-products,
   characterized in that it further comprises the addition, during any step before or after the fermentation step, of a microalgae culture extract.

A “biofuel” is a hydrocarbon, or a mixture of hydrocarbons, which can be used as fuel and which is obtained by using fermentable biomass as starting material. Examples of biofuels include, but are not limited to, ethanol or bioethanol, biodiesel or hydrobiodiesel. In a preferred embodiment of this aspect of the invention, biofuel is ethanol (bioethanol).

The term “fermentable plant biomass” refers to all biomass from plants or parts thereof which accumulates simple sugars or polysaccharides, i.e., polymers whose monomers are monosaccharides repetitively linked by glycosidic bonds and which may be decomposed by hydrolysis of said glycosidic bonds between residues into smaller polysaccharides, as well as into disaccharides or monosaccharides. Examples of this type of biomass include, but are not limited to, trunks, branches, stems, fruits, vegetable residues and waste, etc. Said biomass can come from, for example, but is not limited to, agricultural harvesting (as reeds, grasses, hay or grain, etc.), forestry work and wood industries (such as branches, bark, leaves, stumps, roots, sawdust, etc.), or agricultural residues such as olive stones, almond shells, pinecones, etc. In a preferred embodiment, the fermentable plant biomass referred to by the invention is selected from the list consisting of: biomass rich in fermentable sugars, such as, but not limited to, sugarcane, starchy biomass, such as, but not limited to, wheat grain, or lignocellulosic material, such as, but not limited to, corn straw. In a more preferred embodiment, the fermentable plant biomass is cereal grain. In an even more preferred embodiment, the cereal is corn, wheat, barley or any mixture thereof.

The aqueous medium referred to in step (a) of the method of the invention may consist of, but is not limited to, for example, a mixture of water, enzymes to carry out a first hydrolysis and treatment of biomass, acid-base viscosity corrigents, nutrients, defoamers and/or fermentation salts, and fermentable plant biomass milled and free of sand or other impurities. Preferably, said aqueous medium comprises the enzymes detailed hereunder (cellulases, amylases, glucosidases, etc.), phosphoric acid, sulphuric acid, sodium hydroxide, ammonium hydroxide, calcium chloride, urea, etc. and fermentable biomass, more preferably at a final pH of between 4 and 6 and at temperatures of between 50 and 95° C.

The term “hydrolysis” as used in step (b) of the method of the invention relates to the process which in turn comprises the steps of liquefaction and saccharification of fermentable plant biomass by using hydrolytic enzymes. The process of the invention can be applied to both first and secondgeneration production processes of biofuels, preferably ethanol, and food co-products. Therefore, these two steps of liquefaction and saccharification may be carried out sequentially, for example, but not limited to, in a firstgeneration production process of biofuels, preferably ethanol, and food co-products, or simultaneously, for example, but not limited to, in a secondgeneration production process of biofuels, preferably ethanol, and food co-products. The hydrolysis of the biomass comprised in said aqueous medium can be accomplished by means of enzymatic hydrolysis processes (liquefaction and saccharification) known to those skilled in the art and widely used in the processes for the decomposition of polysaccharides to glucose. These hydrolase enzymes are specific for certain polysaccharides and especially for certain glycosidic bond types. Thus, for example, enzymes that hydrolyze starch, which bonds are α (1→4), cannot decompose cellulose, which bonds are β (1→4), therefore, hydrolytic enzymes used in the hydrolysis process of step (b) of the method of the invention are preferably amylases, cellulases, alpha- and/or beta-glucosidases, endoglucanases, xylanases, cellobiohydrolases, cellobiose dehydrogenase or any mixture thereof. In the case where the liquefaction and saccharification steps are carried out sequentially, a first enzymatic mixture for the liquefaction process and subsequently a second enzyme mixture for the saccharification process are added to the aqueous medium of step (a). In the event that the liquefaction and saccharification steps are carried out simultaneously, a single enzymatic mixture for the liquefaction and saccharification processes is added to the aqueous medium of step (a). Said hydrolysis process is preferably carried out at a temperature of between 50 and 95° C. and at a pH of between 4 and 6.

The fermentation in step (b) of the method of the invention is preferably performed using yeast capable of alcoholic fermentation of the sugars obtained in the hydrolysis process explained in the preceding paragraph. Said yeast is more preferably Saccharomyces cerevisiae. This fermentation process is carried out at, for example, but is not limited to, a temperature of between 28 and 38° C. and at a pH of between 3 and 5.

The hydrolysis and fermentation of step (b) of the method of the invention can be carried out simultaneously, by simultaneously adding hydrolase enzymes and the yeast responsible for the fermentation to the aqueous medium comprising the biomass of step (a), or sequentially, by adding hydrolase enzymes to said medium and, once hydrolysis is completed, by adding the yeast responsible for fermentation. Preferably, in the method of the invention, the hydrolysis and fermentation of step (b) are carried out simultaneously, more preferably the steps carried out simultaneously in step (b) are saccharification and fermentation, and even more preferably under the following conditions: temperature of between 28 and 38° C. and pH of between 3 and 5.

Subsequently, in step (c) of the method of the invention, distillation of the biofuel, preferably ethanol, obtained in step (b) is performed, with a subsequent grinding, drying and final purification thereof. For their part, food co-products are concentrated from the fermentation broth, preferably by decantation and evaporation followed by drying and pelletizing.

The microalgae culture extract used in the method of the invention may originate from a microalgae culture process being carried out in parallel with the method of the invention, or it may be of any other origin. This extract comprises both the microalgae and the culture medium in which they are cultured, which comprises water. Preferably, said culture medium has a fresh or brackish water base, more preferably fresh water. Furthermore, said extract may take various forms, such as but not limited to, liquid, dried, concentrated form, etc. However, since one of the most limiting steps for the viability of the production of microalgae is their harvesting, it is interesting to reduce this limitation using liquid extracts. Hence, in another preferred embodiment, the microalgae culture extract used in the method of the invention is in liquid form.

Fermentable plant biomass may be optionally pre-treated prior to its hydrolysis and/or fermentation in step (b), so that its further enzymatic processing, in hydrolysis, or fermentative processing is optimized. Accordingly, in another preferred embodiment, the microalgae culture extract is added to the aqueous medium of step (a), prior to step (b), to moisten the fermentable plant biomass and so that the simple sugars or polysaccharides comprised therein are more accessible to hydrolytic enzymes which decompose them during the liquefaction and saccharification steps, thus producing fermentable sugars.

Alternatively, the microalgae extract can be added at other points in the method of the invention instead of at the one explained in the preceding paragraph. Thus, in another preferred embodiment, the method of the invention further comprises a step of concentration of the food co-products distilled in step (c) and the microalgae culture extract is added during said step of concentration, or immediately thereafter, such that the protein value of said food co-products is increased. This step of concentration of the food co-products can be accomplished by, for example, but is not limited to, centrifugation, evaporation, drying, etc.

In another preferred embodiment, the method of the invention further comprises a step of microalgae culturing which originates the microalgae culture extract used prior or subsequently to the fermentation step. The nutrients, CO₂ and water used in said culture can come from any source of nutrients, CO₂ and water, but in a more preferred embodiment, the said microalgae culture uses CO₂, water and other nutrients released in the fermentation stage of step (b) or in the product(s) separation of step (c) of the method of the invention. In an even more preferred embodiment, said microalgae culture uses the nitrogen released in the distillation stage of step (c) as a nutrient. Thus, the waste products released in these stages of the process of the invention can be used by reintroducing them into said process. In an even more preferred embodiment, the microalgae culture extract is obtained from the microalgae culture processing and such processing is selected from the list comprising: concentration, homogenization and/or drying.

In another preferred embodiment of the method of the invention, microalgae are selected from the genera: Botryococcus, Neochloris, Nannochloropsis, Phorphyridium, Scenedesmus, Chlorella, Tetraselmis, Spirulina, or any mixture thereof.

The method of the invention may comprise further additional steps related to, for example but not limited to, the pre-treatment of the starting material (fermentable plant biomass) or the processing of the distillates of step (c). Therefore, in another preferred embodiment, the fermentable plant biomass is cleaned of dirt, dust and sand and is pre-treated by means of grinding before step (a), such that its simple sugars and/or polysaccharides are more accessible to the hydrolytic enzymes that decompose them giving way to fermentable sugars. In a more preferred embodiment, the method of the invention further comprises:
d) centrifuging the food co-products distilled in step (c),
e) evaporating the product obtained in the centrifugation of step (d),
f) drying the product obtained in step (e), and
g) pelletizing the product obtained in step (f).

Another aspect of the invention relates to a food co-product obtainable by means of the method of the invention, hereafter “co-product of the invention”.

In the present invention “food co-products” refer to those co-products which are produced, together with the biofuel, preferably ethanol, in the production process of biofuels, preferably ethanol, by fermentable plant biomass fermentation. Said co-products have a valued protein content for animal feed, the result of residual proteins from yeast and fermentable starting biomass, energy, minerals and/or vitamins. This type of co-product may be, but is not limited to, the so-called DDGs formed by the dry mixture of insolubles and solubles from the broth after fermentation. Preferably, the co-product of the invention is a DDG. These co-products have a variable composition depending on different parameters of the production process of biofuels, preferably ethanol, through which they are obtained, such as the starting material, the steps carried out, the physical-chemical conditions under which the process takes place, etc. Therefore, the co-product of the invention has a specific composition which results from the conditions under which the method of the invention is implemented.

As shown in the examples, the co-product of the invention has a high protein content mainly due to the microalgae culture extract used in the method of the invention, hence it is use in the food industry. Thus, another aspect of the invention relates to the use of the co-product of the invention for human consumption and/or animal feed. Another aspect of the invention relates to a food product comprising the co-product of the invention. The food product referred to in the present invention may be for human or animal use, but preferably it is for animal use, more preferably said food product is animal feed.

Throughout the description and claims the term “comprise” and its variations are not intended to exclude other technical features, additives, components or steps. To those skilled in the art, other objects, advantages and characteristics of the invention will become apparent from the specification and practice of the invention. The following examples and figures are provided by way of illustration and are not intended to limit the scope of the present invention.

### DESCRIPTION OF THE FIGURES

FIG. 1. Representative diagram of the method of the invention. The process blocks for the industrial production of ethanol are shown in light gray. The process blocks for microalgae production are shown in dark gray. The possible steps for integration of the two processes according to the method of the invention are marked with double lines. The steps in which streams are produced that may be usable for both processes are explained.

FIG. 2. Representative diagram of the experimental simulation of the method of the invention modifying each of the concentrations and conditions to study the effect. The main techniques used to monitor the processes appear shaded in gray. SSF: simultaneous saccharification and fermentation

FIG. 3. Monitoring the fermentation according to the method of the invention. Results of the analysis of fermentable sugars using HPLC (dashed lines) and of the gravimetric analysis of the ethanol content (solid lines) during the fermentation of wheat (circles), mixtures (10:1) of wheat-Nannochloropsis gaditana (triangles) and mixtures (10:1) of wheat-possible Chlorella (squares) all with (filled-in symbols) and without (empty symbols) the addition of urea to the fermentation broths.

FIG. 4. Monitoring of the fermentation of different microalgae biomass according to the method of the invention. Results of the analysis of fermentable sugars using HPLC (dashed lines) and of the gravimetric analysis of the ethanol content (solid lines) showing ethanol production during the fermentation of wheat (circles), mixtures of wheat-B. braunii (diamonds), mixtures of wheat-N. oleoabundans (triangles) and mixtures of wheat-N. gaditana (squares).

FIG. 5. Monitoring of the fermentation at different ratios of microalgae biomass according to the method of the invention. Results of the analysis of fermentable sugars using HPLC (dashed lines) and gravimetric analysis of the ethanol content (solid lines) showing ethanol production during the fermentation of wheat (circles), mixtures of wheat-Chlorella 10:1 (squares) and wheat-Chlorella 8:1 (triangles).

FIG. 6. Results of the protein fraction analysis. Aminogram and crude protein of the starting biomass and obtained after simultaneous fermentation and saccharification. A) corn DDGS; B) DDGS from the mixture of corn-Nannochloropsis; C) wheat DDGS; D) DDGS from the mixture of wheat—Nannochloropsis.

### EXAMPLES OF EMBODIMENT

The invention will be illustrated hereunder by means of tests performed by the inventors which demonstrate the efficiency of the method of the invention for the production of biofuel, preferably ethanol, and food co-products.

### Materials and Methods

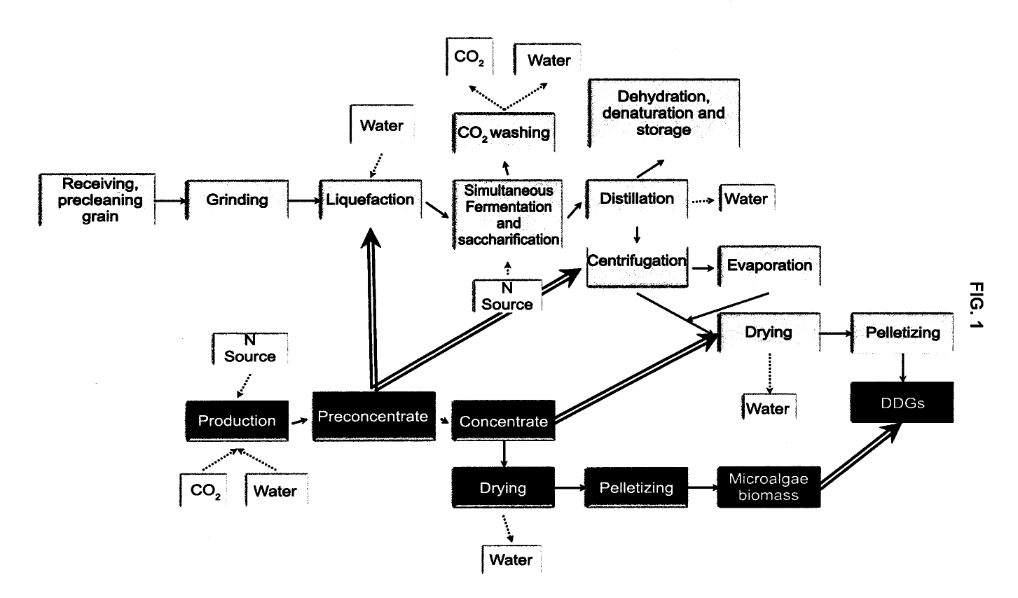

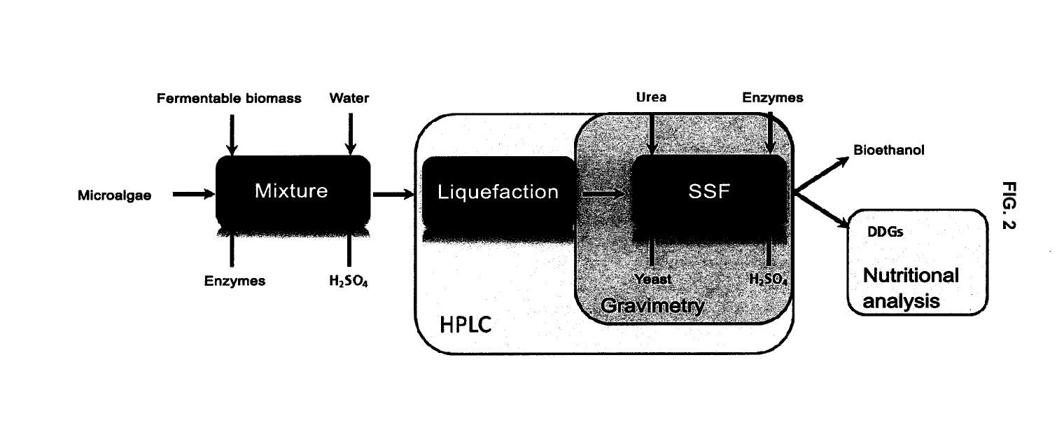

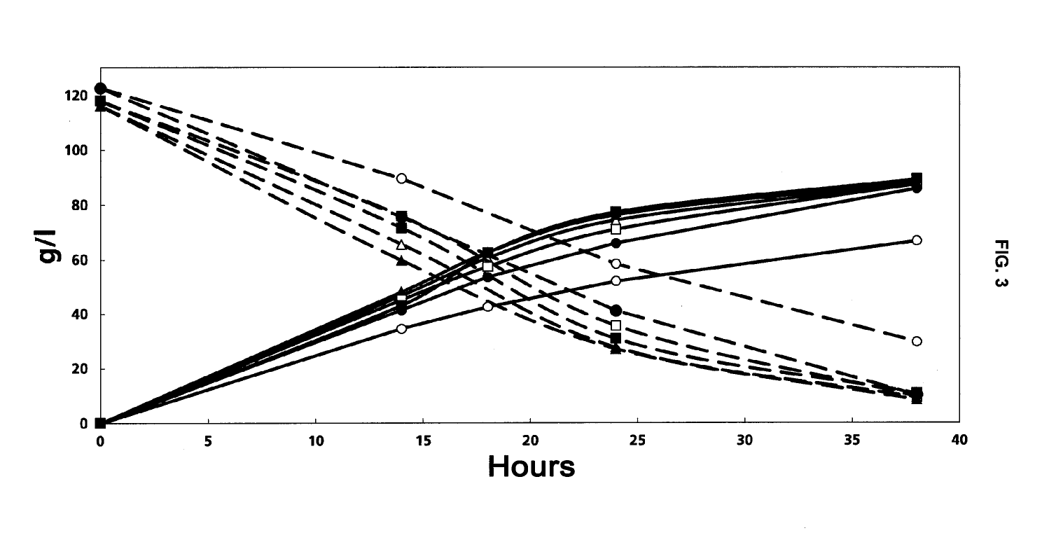

The general outline of most of the tests are shown in FIGS. 1 and 2, modifying each of the concentrations and conditions to study the effect, for example, dosage of enzymes, acids, defoamers, concentration of microalgae, dry matter of the mixture, temperature programme, etc.

Algal biomass was cultured in different lab media, with a fresh water or salt water base (Arnon, BE, F/2, etc.) or in culturing media consisting of agricultural fertilizers. The cultures were aerated with air and/or air enriched with CO₂.

Determining the dry weight of the algal cultures was performed by washing with ammonium formate according to the procedure described by C. J. Zhu & Y. K. Lee (1997). Algal biomass was concentrated to a value of 20% in dry extract by centrifugation at 10,000×G for 10 minutes. The mixture was performed in 100-ml enlenmeyer flasks. Fermentable biomass and algae biomass was added in a ratio of 10:1 (dry weight). The volume required to reach 30% of dry mass in the mixtures was completed with distilled water and the pH was corrected with H₂SO₄ up to a value of between 5.0 and 5.5. The enzyme dosage was performed according to the manufacturer's specifications in a percentage relative to the amount of biomass to hydrolyze (w/w).

The mixture thus prepared was stirred for 30 minutes at a constant temperature of 61° C. Following this phase the temperature was raised to 85° C. in the liquefaction process, keeping the mixture under constant stirring for 3.5 h. After this time the broth was subjected to a simultaneous saccharification and fermentation (SSF) in anaerobiosis at 30° C., after pH adjustment with sulphuric acid to 3.8. To do this, enzymes and defoamer were added at concentrations recommended by the manufacturers. Urea at a final concentration of 0.053% was added as a nitrogen source. Once this fermentation medium was prepared, ethanologenic commercial yeast inoculum was added at a final concentration of 10⁷ cfu/ml.

Ethanol production was monitored by gravimetry and gas chromatography with flame ionization detector (GC-FID). The fermentable sugar content was quantified by high performance liquid chromatography (HPLC) using external calibration standards.

The content of dry mass was determined by drying in an oven at 103° C. to constant weight (usually 8 h), ash by incineration in a muffle furnace at 550° C., crude protein (Kjeldahl), crude fibre (Ankom), the crude fat by Soxlet, all according to procedures described in AOAC, 2000.

The carbohydrate content was estimated using the Anthrone method.

The determination of macronutrients (calcium, magnesium, sodium and potassium) and micronutrients (copper, zinc, iron and manganese) was performed using atomic absorption spectroscopy.

### Example 1

### Simultaneous Saccharification and Fermentation of Wheat, Mixtures (10:1) of Wheat—Nannochloropsis Gaditana and Mixtures (10:1) of Wheat-Possible Chlorella. All with and without the Addition of Urea as a Nitrogen Source for the Fermentation

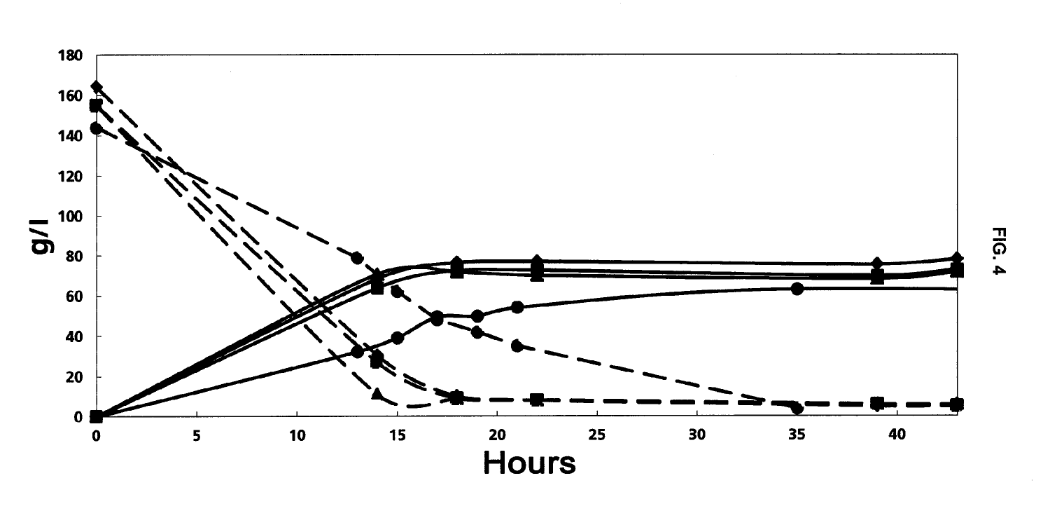

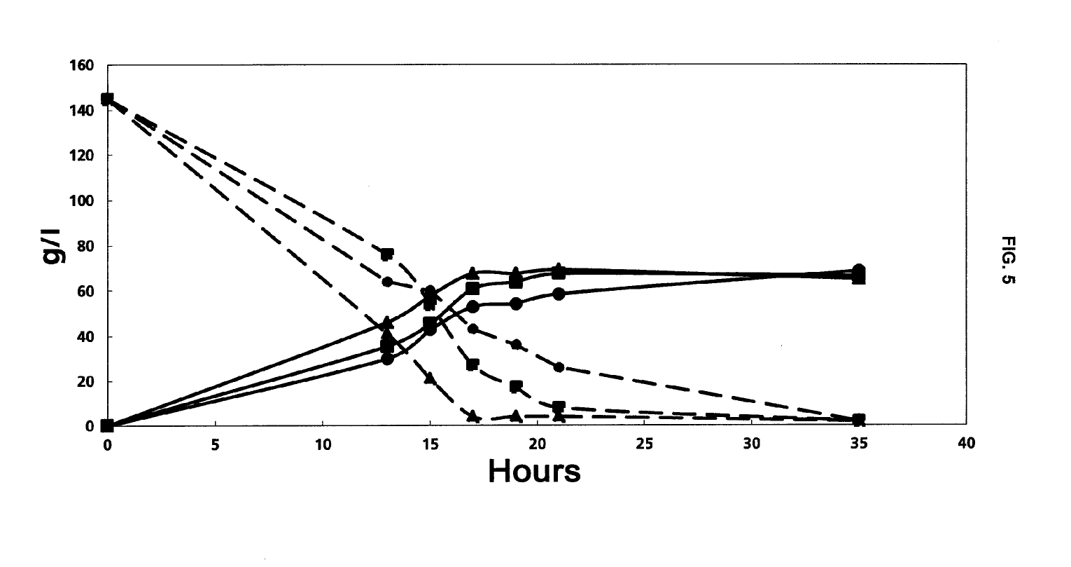

Following the general procedure described above, wheat and wheat-microalgae mixtures with a 10:1 ratio were simultaneously fermented and saccharificated. The fermentation broths reached 30% in dry mass. The enzymes were dosed at concentrations recommended by the manufacturer, as occurs in a plant for the industrial production of ethanol from cereal. The results are shown in FIG. 3. As shown in this figure, all mixtures assayed containing algae culture extract accelerate the production of ethanol as well as the assimilation of sugars by yeasts. It can also be seen that the addition of microalgae culture extract keeps up the ethanol productivity and assimilation of sugars at levels higher than those obtained without adding a nitrogen source (urea) to the fermentation medium.

### Example 2

### Saccharification and Fermentation of Several Species of Microalgae and Wheat

This example shows the values obtained with several samples of algae (Botryococcus braunii, Neochloris oleoabundans y Nannochloropsis gaditana).

Following the general procedure described above wheat and wheat-microalgae mixtures at a 4:1 ratio were first saccharificated and subsequently fermented. The saccharification was performed with enzymes dosed at 1%. The fermentation broths reached 25% by dry weight and were not supplemented with urea as a nitrogen source.

The results are shown in FIG. 4. As shown in this figure all the mixtures tested accelerate the production of ethanol as well as the assimilation of sugars by yeasts. Yields vary between the different mixtures, reaching different ethanol productivity levels, also depending on the initial contribution in carbohydrates of each microalgae species.

### Example 3

### Simultaneous Saccharification and Fermentation at Various Wheat-Chlorella sp. Ratios

This example shows the results obtained after simultaneous saccharification and fermentation of wheat (control) and two mixtures (10:1 and 8:1) of wheat and Chlorella sp. biomass. The saccharification enzymes were dosed in excess (1%) compared to the dry mass of the fermentation broth (20% in this example).

As shown in FIG. 5, at these ratios and considering the small amount of fermentable carbohydrates provided—in this case, the microalgae biomass (6.3% according to the Anthrone method)—the final values of ethanol coincide with the wheat control sample and only an acceleration of the reaction is noted.

### Example 4

### Nutritional Characterization of the Ethanol Co-Products Obtained Using Wheat, Corn and Wheat and Microalgae Mixtures and Corn and Microalgae Mixtures as Raw Material

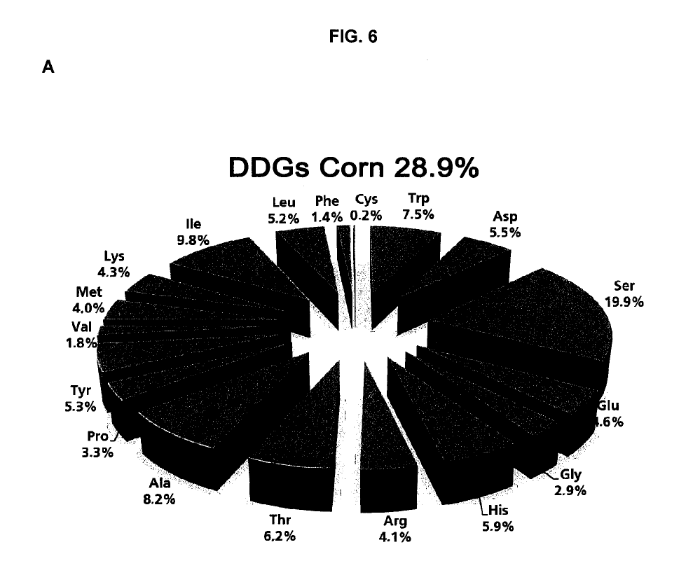

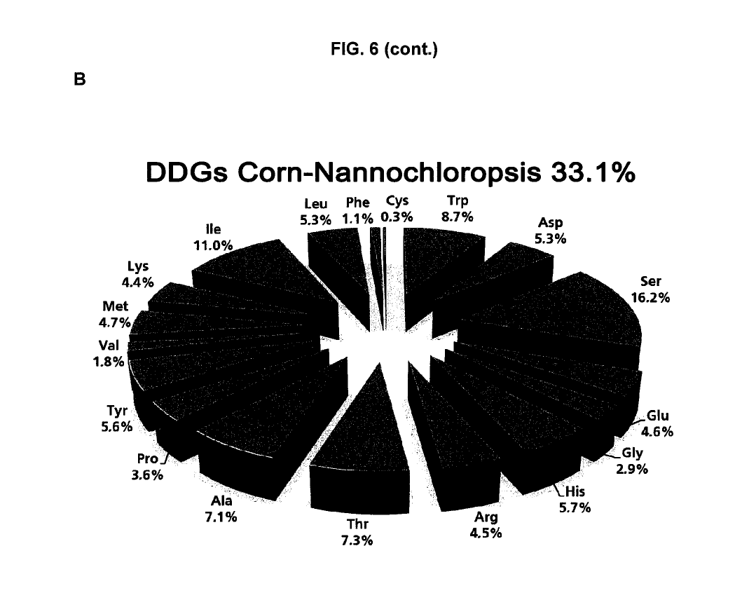

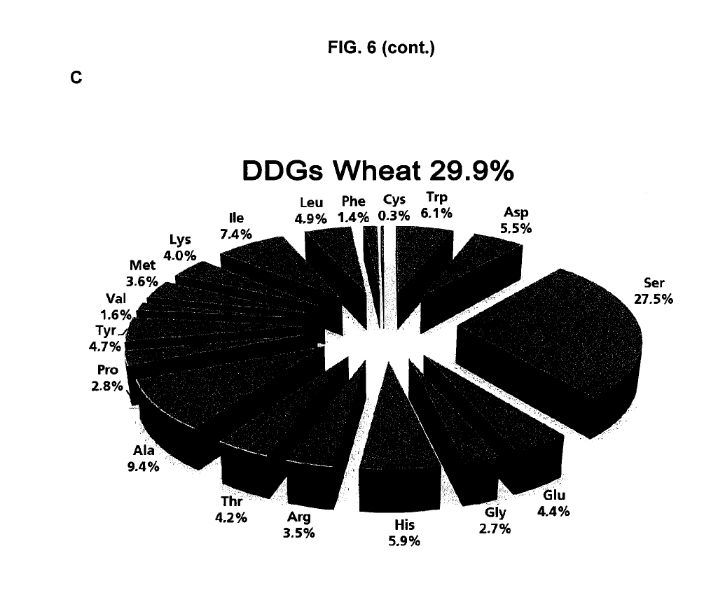

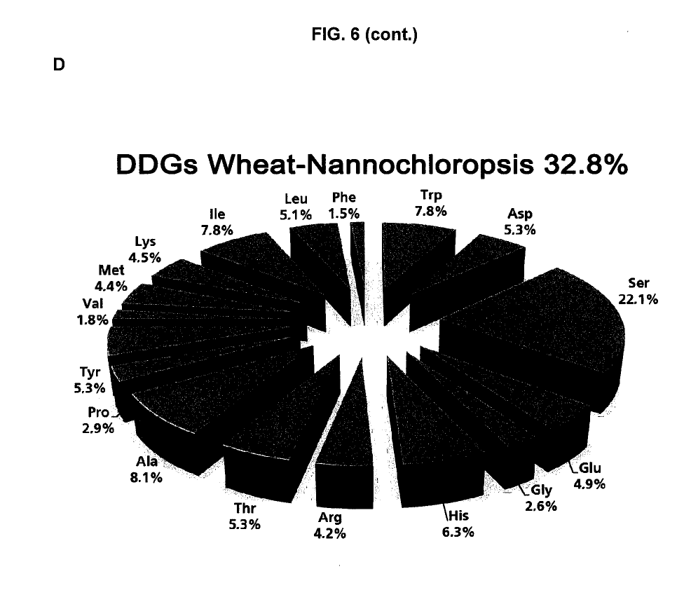

Co-products of the simultaneous saccharification and fermentation of cereal grain and Nannochloropsis gaditana biomass in a 10:1 ratio were nutritionally analyzed. As shown in FIG. 6, the percentages of crude protein based on dry mass, increase about 3% as expected based on the ratio used. The resulting aminograms show that the ethanol co-products obtained by mixing with Nannochloropsis additionally enhance protein content, the percentage of amino acids lacking in most cereal-based diets (e.g., lysine, tryptophan), and thus are valuable for the formulation of compound feeds.

1. Method for the production of a biofuel and food co-products from fermentable plant biomass, comprising:
a. preparing an aqueous medium comprising said biomass,
b. simultaneously or sequentially hydrolyzing and fermenting the biomass comprised in the aqueous medium of step (a), and
c. distilling the products obtained in step (b) in order to obtain biofuel and food co-products,
characterized in that it further comprises the addition, at any step before or after the fermentation stage, of a microalgae culture extract and wherein the hydrolysis of step (b) comprises the steps of liquefaction and saccharification by using hydrolytic enzymes.

2. The method according to claim 1, wherein the fermentable plant biomass is selected from the list consisting of: biomass rich in fermentable sugars, starchy biomass or lignocellulosic material.

3. The method according to claim 1, wherein the microalgae culture extract is liquid.

4. The method according to claim 1, wherein the microalgae culture extract is added to the aqueous medium of step (a).

5. The method according to claim 1, further comprising a step of concentration of the food co-products distilled in step (c) and wherein the microalgae culture extract is added during the said concentration step or immediately thereafter.

6. The method according to claim 1, further comprising a step of microalgae culturing from which the microalgae culture extract is obtained.

7. The method according to claim 6, wherein the microalgae culture uses CO₂, water and other nutrients released in the fermentation stage of step (b).

8. The method according to claim 6, wherein the microalgae culture uses the nitrogen released in the distillation stage of step (c) as a nutrient.

9. The method according to claim 6, wherein the microalgae culture extract is obtained from the microalgae culture processing, and wherein said processing is selected from the list comprising: concentration, homogenization or drying.

10. The method according to claim 1, wherein the microalgae are selected from the genera: Botryococcus, Neochloris, Nannochloropsis, Phorphyridium, Scenedesmus, Chlorella, Tetraselmis or Spirulina.

11. The method according to claim 1, wherein the fermentable plant biomass is pre-treated by grinding before step (a).

12. The method according to claim 1, further comprising:
d. centrifuging the food co-products distilled in step (c),
e. evaporating the product obtained in the centrifugation of step (d),
f. drying the product obtained in step (e), and
g. pelletizing the product obtained in step (f).

13. Food co-product obtainable by the method according to claim 1.

14. Food product comprising the co-product of claim 13.

15. (canceled)
